(19) United States
(12) Patent Application Publication
(10) Pub. No.: US 2016/0303081 A1
(43) Pub. Date: Oct. 20, 2016

(54) INHIBITORS OF BETA1-INTEGRIN AND METHODS OF USE

(71) Applicant: The Texas A&M University System, College Station, TX (US)

(72) Inventors: Stephen Safe, College Station, TX (US); Syng-Ook Lee, College Station, TX (US)

(73) Assignee: The Texas A&M University System, College Station, TX (US)

(21) Appl. No.: 15/130,795

(22) Filed: Apr. 15, 2016

Related U.S. Application Data
(60) Provisional application No. 62/149,289, filed on Apr. 17, 2015.

(51) Int. Cl. A61K 31/404

(52) U.S. Cl. CPC A61K 31/404

(57) ABSTRACT

The present disclosure provides compositions and methods for inhibiting β1-integrin expression and functionality, and for addressing any condition characterized by increased β1-integrin levels, through inhibition of NR4A1 (TR3) in the cell.

### CROSS-REFERENCE TO RELATED APPLICATION

This application claims the benefit of Provisional Application No. 62/149,289, filed Apr. 17, 2015, which is expressly incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is TAMUS155757_ST25.txt. The text file is 4 KB, was created on Apr. 15, 2016, and is being submitted via EFS-Web with the filing of the specification.

### FIELD OF THE INVENTION

The present disclosure relates to compositions and methods for inhibiting β1-integrin expression and functionality, and addressing any condition characterized by increased β1-integrin levels, through inhibition of NR4A1 (TR3).

### BACKGROUND

Cell adhesion and attachment are essential for tissue integrity and cellular homeostasis. The heterodimeric integrin cell surface receptors play a critical role in these critical cellular processes. There are 18 different α subunits and 8 different β subunits that form 24 α,β-integrin receptor heterodimers. The large 12-member β1-integrin sub-group binds to extracellular matrix (ECM) molecules such as collagen, laminin, fibronectin, tenascin C and vitronectin. Interactions of the integrin receptors with ECM components activate multiple intracellular pathways and also induce crosstalk with other signaling systems including the epidermal growth factor receptor (EGFR) and other receptor tyrosine kinases. The functions of integrin heterodimers are highly tissue-specific and many human pathologies also involve integrin signaling. Considering its pivotal role in critical cellular function, β1-integrin has been the focus as a potential target in numerous clinical trials for the potential treatment of a variety of conditions and diseases, including cancer, thrombosis, inflammatory conditions, autoimmune diseases, arthritis, age-related macular degeneration, stroke, and a variety of cancers.

For example, β1-integrin has been identified as one of the most important integrin receptors in tumorigenesis with prognostic significance and multiple pro-oncogenic functions in several tumor types. β1-integrin is highly expressed in most tumors and is associated with a negative prognostic significance such as overall and disease free survival, recurrence, and metastasis for head and neck and squamous cell carcinoma, melanoma, lung, breast, prostate, laryngeal and pancreatic cancers. There is increasing evidence that β1-integrin is a pivotal gene involved in tumor growth, survival, adhesion, migration and invasion of cancer cells, and the dominance of one or more of these pathways is dependent on the cell and tumor type, and the differential expression of integrin subfamily members. Results of in vitro and transgenic animal model studies show that β1-integrin-containing receptors play an important role in mammary tumor initiation, progression and metastasis. For example, mice that express the polyoma virus middle T antigen under control of the MMTV promoter rapidly develop mammary tumors that metastasize to the lung, and selective knockout of β1-integrin in luminal epithelial cells blocks tumor formation. In vitro studies with mammary tumor cells derived from the transgenic mice showed that loss of β1-integrin inhibited proliferation and this was due, in part, to inactivation of focal adhesion kinase (FAK), an immediate downstream target of β1-integrin. Additionally, β1-integrin mRNA and protein are overexpressed in pancreatic tumors. β1-integrin silencing by RNA interference (RNAi) in pancreatic cancer cells decreases cell adhesion, migration and invasion, and downregulation of genes such as MMP-2 and MMP-9. Knockdown of β1-integrin in Colo-357 cells by RNAi also decreases expression of α2-, α3-, α5- and αv-integrins, which form heterodimers with β1-integrin and bind collagen (α2-), laminin (α3-), and fibronectin (α5- and αv-). α silencing by RNA interference (RNAi) also decreases tumor growth and metastasis. Similar results have been observed in vivo using β1-integrin antibodies.

There have been clinical trials on over 250 anti-integrin drugs for treatment of multiple diseases including thrombosis, inflammatory conditions, autoimmune disease, arthritis, age-related macular degeneration, stroke and cancer. The drugs typically act as inhibitors (e.g. antibodies, peptides, small molecules) of β1-integrin signaling. For example, such proposed β1-integrin inhibitors generally target key binding sites on β1-integrin. However, these approaches have not been entirely successful in providing efficacious therapy without and non-toxic cancer chemotherapy.

Accordingly, despite the advances in the art, there remains a recognized need for improved therapies that target β1-integrin function, but which are non-toxic and simple to manufacture. The invention set forth in this disclosure addresses this need and provides further advantages related thereto.

### SUMMARY

This summary is provided to introduce a selection of concepts in a simplified form that are further described below in the Detailed Description. This summary is not intended to identify key features of the claimed subject matter, not is it intended to be used as an aid in determining the scope of the claimed subject matter.

In one aspect, the disclosure provides a method of inhibiting β1-integrin expression in a cell. The method comprises inhibiting NR4A1 in the cell.

In some embodiments, inhibiting NR4A1 in the cell comprises reducing the level of functional NR4A1 in the cell. In other embodiments, inhibiting NR4A1 in the cell comprises contacting the cell with an NR4A1 ligand that exhibits antagonist activity. In some embodiments, the NR4A1 antagonist is a C-substituted diindolylmethane compound (C-DIM). In some embodiments, the C-DIM is 1,1-bis(3′-indolyl)-1-(p-hydroxyphenyl)methane (DIM-C-pPhOH) or a related compound where the -pPhOH group contains other substituents or is replaced by another aromatic group.

In some embodiments, the cell is a cancer cell. In some embodiments, the cell is a pancreatic cancer cell, a colon cancer cell, or a breast cancer cell.

In some embodiments, the cell is contacted with the NR4A1 antagonist in vitro. In other embodiments, the cell is contacted with the NR4A1 antagonist in vivo by administering an effective amount of the NR4A1 antagonist to a subject. In some embodiments, the NR4A1 antagonist is administered with a pharmaceutically acceptable carrier. In some embodiments, the administering comprises topical administration, oral administration, intravenous injection, intraperitoneal injection, intramuscular injection, intranasal administration, transdermal administration, or rectal administration. In some embodiments, the subject is a mammal.

In another aspect, the disclosure provides a method of treating a disorder treatable by inhibiting β1-integrin in one or more cells of a subject with the disorder, comprising administering a therapeutically effective amount of an NR4A1 inhibitor to the subject.

In some embodiments, the NR4A1 inhibitor comprises a ribonucleic acid that corresponds to at least a portion of the NR4A1 mRNA sequence set forth in SEQ ID NO:1. In some embodiments, the NR4A1 inhibitor comprises a ribonucleic acid that corresponds to at least a portion of the NR4A1 mRNA sequence set forth in SEQ ID NO:1. In some embodiments, the portion of the NR4A1 mRNA sequence is at least 15 contiguous nucleotides of the sequence set forth in SEQ ID NO:1. In some embodiments, the NR4A1 inhibitor is an NR4A1 antagonist. In some embodiments, the NR4A1 antagonist is a C-substituted diindolylmethane compound (C-DIM). In some embodiments, the C-DIM is 1,1-bis(3′-indolyl)-1-(p-hydroxyphenyl)methane (DIM-C-pPhOH) or a related compound where the -pPhOH group contains other substituents or is replaced by another aromatic group.

In some embodiments, the disorder is cancer, thrombosis, colitis, Crohn's disease, inflammatory bowel disease, multiple sclerosis, rheumatoid arthritis immunosuppression disorder, arthritis, asthma, stroke, restenosis, rhinitis, and osteoporosis. In some embodiments, the cancer is a pancreatic cancer, a colon cancer, or a breast cancer. In some embodiments, treating the cancer comprises preventing or inhibiting the migration capacity of cancer cells in the subject. In some embodiments, treating the cancer comprises preventing or inhibiting the invasion capacity of cancer cells in the subject. In some embodiments, treating the cancer comprises preventing or inhibiting the growth of cancer cells in the subject. In some embodiments, the subject is a mammal selected from the group consisting of rat, mouse, guinea pig, dog, cat, cow, horse, sheep, pig, primate, and human. In some embodiments, the NR4A1 inhibitor is administered with a pharmaceutically acceptable carrier. In some embodiments, the administering comprises topical administration, oral administration, intravenous injection, intraperitoneal injection, intramuscular injection, intranasal administration, trans-dermal administration, or rectal administration.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a schematic illustration of the knockdown of NR4A1 or application of C-DIM/NR4A1 antagonists result in inhibition of NR4A1-dependent growth and survival pathway.

FIGS. 2A and 2B graphically illustrate that C-DIMs are NR4A1 antagonists and receptor ligands. DIM-C-pPhOH (2A) and DIM-C-pHCO₂Me (2B) decrease NR4A1-dependent transactivation and bind the ligand binding domain of NR4A1.

FIGS. 3A and 3B illustrate that the knockdown of NR4A1 decreases invasion of MiaPaCa-2 (3A) and Panc1 (3B) cells in a Boyden Chamber assay. Significant inhibition with p<0.001 indicated with “#”.

FIGS. 4A and 4B illustrate that DIM-C-pPhOH decreases pancreatic cancer cell invasion in a Boyden Chamber assay. Significant inhibition with p<0.001 indicated with “#”.

FIGS. 5A and 5B illustrate that knockdown of NR4A1 by RNA interference resulted in decreased expression of β1-integrin in Panc1 cells. (5A) is a heatmap indicating mRNA microarray results. (5B) graphically illustrates real-time PCR results. Significant inhibition with p<0.001 indicated with “#”.

FIGS. 6A and 6B illustrate that NR4A1 silencing decreases β1-integrin and related gene products in MiaPaCa-2 (6A) and Panc1 (6B) cells.

FIG. 7 illustrates that DIM-C-pPhOH NR4A1 antagonist (20 μM) also decreases β1-integrin and related gene products in pancreatic cancer cells.

FIGS. 8A and 8B illustrate that knockdown of NR4A1 by RNA interference decreases MiaPaCa-2 (8A) and Panc1 (8B) cell adhesion to plates coated with human fibronectin. Similar results were observed with siRN4A1-2. Significant inhibition with p<0.001 indicated with “#”.

FIG. 9 graphically illustrates that DIM-C-pPhOH (20 μM) decreases pancreatic cancer cell adhesion to human fibronectin-coated plates in an adhesion assay. Significant inhibition with p<0.001 indicated with “#” and significant inhibition with p<0.005 indicated with “***”.

FIG. 10 illustrates tumor lysates from an L3.6pL orthotopic xenograft experiment in vivo, which show that DIM-C-pPhOH (30 mg/kg/d) decreases expression of β1-integrin and α5-integrin compared to corn oil control. The tumor lysate from two animals is shown.

FIG. 11 illustrates knockdown of NR4A1/TR3 by RNA interference (siTR3) decreases β1-integrin expression in MDA-MB-231 and SKBR3 breast cancer cell lines. Similar results were observed in MCF-7 cells.

FIG. 12 illustrates that treatment of breast cancer cells with the NR4A1 antagonist DIM-C-pPhOH decreases β1-integrin expression.

FIG. 13 illustrates that treatment of RKO and SW480 colon cancer cells with the NR4A1 antagonist DIM-C-pPhOH decreases β1-integrin expression.

FIG. 14 is a series of photographs illustrating that the knockdown of NR4A1 (TR3) decreases migration/invasion of MDA-MB-231 and SKBR3 cells.

FIG. 15 illustrates that the NR4A1 antagonist DIM-C-pPhCO₂ME inhibits migration/invasion of MDA-MB-231 cells.

FIG. 16 illustrates that the NR4A1 antagonist DIM-C-pPhCO₂ME inhibits migration/invasion of SKBR3 cells.

FIG. 17 illustrates that knockdown of β1-integrin by RNA interference (siβ1-integrin) decreases migration/invasion of SKBR3 cells. Similar results were obtained in MDA-MB-231 cells.

### DETAILED DESCRIPTION

The present disclosure is based on the inventor's surprising discovery NR4A1 (also referred to as “TR3”) is a strong regulator of β1-integrin expression. NR4A1 and related receptors NR4A2 (Nurr1) and NR4A3 (Nor1) are immediate early genes induced by multiple stimuli/stressors and play essential roles in metabolic processes, inflammation, vascular function, steroidogenesis, and the central nervous system functionality. As described in more detail below, the present inventors discovered that inhibition of the orphan nuclear receptor NR4A1 (also referred to as TR3) results in the marked downregulation of β1-integrin. While investigating the effects of NR4A1 silencing using a Sentrix Human V.3 HT12 beadchip array, the inventors discovered that β1-integrin expression is regulated by NR4A1. The inventors developed a series of diindolylmethane analog compounds with modifications at the bridge carbon (C-substituted DIMs, or “C-DIMs”) that inactivate nuclear NR4A1. Through subsequent assays, the NR4A1 antagonistic C-DIM compounds were demonstrated to result in decreased expression of β1-integrin and, thus, represent a novel class of β1-integrin inhibitors.

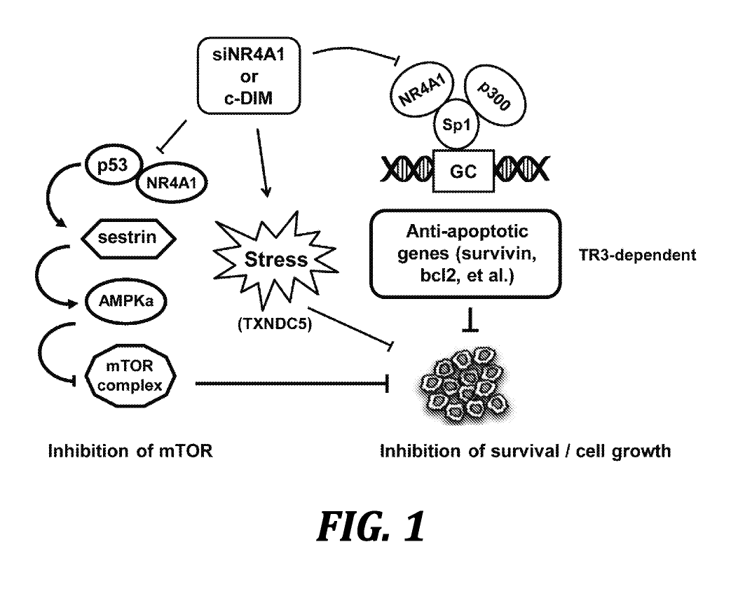

As noted above, β1-integrin expression plays a key role in multiple diseases including various cancers, and β1-integrin inhibitors generally target critical β1-integrin binding sites with diverse drugs, including antibodies, to block β1-integrin signaling and function. The present disclosure provides a novel approach for inhibiting β1-integrin expression through its upstream regulation and thereby β1-integrin-regulated pathways. This disclosure provides various advantages to the prior approaches to regulate β1-integrin. The present disclosure provides the novel demonstration that β1-integrin expression is regulated by the orphan nuclear receptor NR4A1 and, as described in more detail below, knockdown of the receptor by RNA interference decreases expression of β1-integrin and β1-integrin-regulated pathways. Additionally, as described in more detail below, NR4A1 antagonists such as C-DIMs, including DIM-C-pPhOH, ultimately have the effect of decreasing β1-integrin expression in cancer cells. This results in decreased β1-integrin-regulated genes and pro-oncogenic pathways. NR4A1 antagonists also provide an advantage in that they also target other pathways relevant to cancer therapeutics (see FIG. 1), and could appropriately be combined with other drugs for a multi-pronged approach to therapy. Furthermore, with specific reference to C-DIMs as a novel class of β1-integrin regulators, the antagonists of the NR4A1/β1-integrin have attractive advantages for specific application in therapeutic approaches. For example, C-DIMs provide the advantage of allowing rapid, inexpensive, and relatively simple synthesis. Moreover, in sharp contrast to many previously investigated direct β1-integrin antagonists, there is substantial evidence that C-DIMs as a class are relatively non-toxic. C-DIMs have been shown to be relatively non-toxic to non-transformed cells and protect against inflammation of neuronal and vascular cells. See, e.g., Calabro, P., et al., “Inhibition of tumor necrosis factor-a-induced endothelial cell activation by a new class of PPARγ agonists: an in vitro study showing receptor-independent effects,” J. Vascular Res. 42:509-516 (2005), and Carbone, D. L., et al., Suppression of 1-methyl-4-phenyl-1,2,3,6-tetrahydropyridine-induced nitric oxide synthase 2 expression in astrocytes by a novel diindolylmethane analog protects striatal neurons against apoptosis,” Mol. Pharmacol. 75:35-43 (2009), each of which are incorporated herein by reference in their entireties. In tumor growth inhibition studies, C-DIMs inhibit tumor growth but do not induce toxicity. See, e.g., Cho, S. D., et al., “Nur77 agonists induce proapoptotic genes and responses in colon cancer cells through nuclear receptor-dependent and independent pathways,” Cancer Res. 67:674-683 (2007) and Lee, S. O., et al., “Inactivation of the orphan nuclear receptor TR3/Nur77 inhibits pancreatic cancer cell and tumor growth,” Cancer Res. 70:6824-36 (2010), each of which are incorporated herein by reference.

In accordance with the foregoing, in one aspect the present disclosure provides a method of inhibiting β1-integrin expression in a cell, comprising inhibiting NR4A1 in the cell.

As used herein, the term inhibiting NR4A1 encompasses any action that results in lower levels of functionalized NR4A1 protein. Functionalized NR4A1 protein is NR4A1 protein that is capable of initiating or promoting transcription of β1-integrin.

In some embodiments, inhibiting NR4A1 comprises reducing the expression of NR4A1. In some embodiments, inhibiting NR4A1 comprises reducing the levels of NR4A1 mRNA transcripts, for example by RNA interference, to prevent or reduce the levels of transcribed NR4A1 protein. RNA interference can be accomplished according to standard protocols, wherein RNA constructs corresponding to (e.g., able to hybridize to) sections of the NR4A1 transcript, such as the sequence set forth in SEQ ID NO:1, are administered to the cell. Typically, constructs as small as about 18-25 nucleotides or so that hybridize to the NR4A1 transcript can trigger the cell-directed degradation of the resulting double stranded RNA constructs, thus preventing translation from the mRNA template. Accordingly, in some embodiments, the method comprises contacting the cell with RNAi constructs that hybridize to at least 18 nucleotides within SEQ ID NO:1, or to a nucleic acid sequence with at least 80%, 85%, 90%, 91%, 92%, 93%, 94%, 95%, 96%, 97%, 98%, 99% identity to SEQ ID NO:1. In some embodiments, the at least 18 nucleotides is a contiguous subsequence of SEQ ID NO:1 or a sequence with at least 80%, 85%, 90%, 91%, 92%, 93%, 94%, 95%, 96%, 97%, 98%, 99% identity to SEQ ID NO:1. The subsequence can be any contiguous subsequence of SEQ ID NO:1 or a sequence with at least 80%, 85%, 90%, 91%, 92%, 93%, 94%, 95%, 96%, 97%, 98%, 99% identity to SEQ ID NO:1.

In some embodiments, inhibiting NR4A1 comprises the binding of a ligand to the NR4A1 protein. A ligand is a molecule that has binding affinity to the NR4A1. In some embodiments, the ligand has antagonistic activity, namely the ligand has reduced or no efficacy in stimulating the cognate function of the receptor (e.g., an antagonist ligand). In some embodiments, the antagonist ligand blocks the constitutive function of the receptor and its ability for stimulatory, cognate ligands to bind to the NR4A1 protein and to activate NR4A1-dependent genes.

Compounds useful as NR4A1 inhibitors (e.g., as antagonists) in the methods and compositions of the invention include C-DIM compounds. As used herein, the term “C-DIM compounds” refers to the C-DIM compounds described in U.S. Pat. No. 7,232,843, which is hereby incorporated by reference in its entirety, and is described briefly below.

C-DIM compounds (“C-substituted DIMS” or “C-DIM compounds”) have modifications at the diindolylmethane bridge carbon. These compounds can be symmetrical or asymmetrical, depending on whether a single indole precursor is used in the synthesis (leading to a symmetrical C-substituted DIM, or if two different indole precursors were used (leading to an asymmetrical C-substituted DIM). The C-substituted DIMS are generally represented by the following structure:

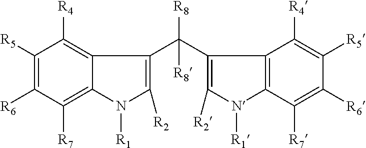

where R₁, R₂, R₄, R₅, R₆, R₇, R₁′, R₂′, R₄′, R₅′, R₆′, and R₇′, individually and independently, is hydrogen, or a substituent selected from the group consisting of a halogen, a nitro group, and a linear or branched alkyl or alkoxy group of about one to about ten carbons, preferably of about one to about five carbons, said compound having at least one substituent. The halogen is selected from the group consisting of chlorine, bromine, and fluorine.

In a preferred embodiment of the C-DIM compounds, R₁, R₂, R₄, R₆, R₇, R₁′, R₂′, R₄′, R₆′, and R₇′ are hydrogen, R₅ and R₅′ are a halogen selected from the group consisting of chlorine, bromine and fluorine. Accordingly, preferred C-DIM compounds include 5,5′-dichloro-diindolylmethane, 5,5′-dibromo-diindolylmethane, and 5,5′-difluoro-diindolylmethane.

Additional preferred C-DIM compounds include compounds wherein R₁, R₂, R₄, R₆, R₇, R₁′, R₂′, R₄′, R₆′, and R₇′ are hydrogen, R₅ and R₅′ are an alkyl or alkoxyl having from one to ten carbons, and most preferably one to five carbons. These include, but are not limited to 5,5′-dimethyl-diindolylmethane, 5,5′-diethyl-diindolylmethane, 5,5′-dipropyl-diindolylmethane, 5,5′-dibutyl-diindolylmethane and 5,5′-dipentyl-diindolylmethane. These also include, but are not limited to, 5,5′-dimethoxy-diindolylmethane, 5,5′-diethoxy-diindolylmethane, 5,5′-dipropyloxy-diindolylmethane, 5,5′-dibutyloxy-diindolylmethane, and 5,5′-diamyloxy-diindolylmethane.

Additional preferred C-DIM compounds include compounds wherein R₂, R₄, R₅, R₆, R₇, R₂′, R₄′, R₅′, R₆′, and R₇′ are hydrogen, R₁ and R₁′ are an alkyl or alkoxyl having from one to ten carbons, and most preferably one to five carbons. Such useful compounds include, but are not limited to, N,N′-dimethyl-diindolylmethane, N,N′-diethyl-diindolylmethane, N,N′-dibutyl-diindolylmethane, and N,N′-dipentyl-diindolylmethane.

In yet another preferred embodiment, R₁, R₄, R₅, R₆, R₇, R₁′, R₄′, R₅′, R₆′, and R₇′ are hydrogen, and R₂ and R₂′ are alkyl of one to ten carbons, and most preferably one to about five carbons. Such compounds include, but are not limited to, 2,2′-dimethyl-diindolylmethane, 2,2′-diethyl-diindolylmethane, 2,2′-dipropyl-diindolylmethane, 2,2′-dibutyl-diindolylmethane, and 2,2′-dipentyl-diindolylmethane.

In another embodiment, R₁, R₂, R₄, R₆, R₇, R₁′, R₂′, R₄′, R₆′, and R₇′ are hydrogen, and R₅ and R₅′ are nitro.

In each of the above embodiments, R₈ and R₈′ are each independently selected from the group consisting of hydrogen, a linear alkyl group containing one to about ten carbon atoms, a branched alkyl group containing one to about ten carbon atoms, a cycloalkyl group containing one to about ten carbon atoms, and an aryl group. At least one of R₈ and R₈′ are not hydrogen. A preferred embodiment of C-substituted DIMs includes when R₁, R₂, R₁′, and R₂′ are each individually hydrogen or methyl; R₄, R₅, R₆, R₇, R₄′, R₅′, R₆′, and R₇′ are each hydrogen; and R₈ and R₈′ are each individually hydrogen, methyl, C₆H₅, C₆H₄OH, C₆H₄CH₃, C₆H₄CF₃, C₁₀H₇, C₆H₄C₆H₅, or C₆H₄OCH₃. Depending on the nature of the two indole subunits, and of R₈ and R₈′, it is possible for the bridging carbon atom to be a chiral center (a carbon atom with four different substituents attached). If a chiral center exists, then the resulting C-substituted DIM would consist of two mirror image enantiomers, each of which is optically active. Resolution of the mixture using a chiral chromatography column or other means would result in the isolation of purified or pure enantiomer products. The different enantiomers may prove to have different biological activities.

In some embodiments, at least one of R₈ and R₈′ is not hydrogen. In some embodiments, at least one of R₈ and R₈′ is pPhX, where X is another substituent such as CF₃, Br, F, t-Butyl, OCH₃, N-dimethylamino, H, OH, C₆H₅, CN, CH₃, Cl, I and CO₂Me, in the ortho-, meta- or para-orientations. In some embodiments, the C-DIM is 1,1-bis(3′-indolyl)-1-(p-hydroxyphenyl)methane (DIM-C-pPhOH). In some embodiments, the C-DIM is (DIM-C-pPhOH) with additional substituents on the pPhOH group.

The synthesis of the substituted indole-3-carbinol (I3C) derivatives from the commercially-available substituted indoles is a convenient method for preparation of these compounds. The C-DIM compounds can also be prepared by condensation of formaldehyde with substituted indoles; however, a disadvantage of the latter reaction is the formation of by-products which will complicate purification of the desired C-DIM. The compounds can be synthesized by dimethylformamide condensation of a suitable substituted indole to form a substituted indole-3-carboxaldehyde. Suitable substituted indoles include those indoles having substituents at R₁, R₂, R₄, R₅, R₆ and R₇ positions. These include, but are not limited to 5-methoxy, 5-chloro, 5-bromo, 5-fluoro, 5-methyl, 5-nitro, N-methyl, and 2-methyl indoles. The substituted indole 3-aldehyde product is treated with a suitable alcohol such a methanol and solid sodium borohydride to reduce the aldehyde moiety to give substituted I3Cs. C-DIMs are prepared by condensing the substituted indole-3-carbinol products. This may be achieved, for example, by treatment with a phosphate buffer having a pH of about 5.5. Use of a single indole starting material will lead to symmetrical products, while use of two different indole starting materials will lead to asymmetrical products.

The preparation and characterization of representative C-DIM compounds is described in U.S. Pat. No. 7,232,843, incorporated herein by reference in its entirety.

As described herein, the NR4A1 has been demonstrated to be a strong regulator of β1-integrin, and both NR4A1 and β1-integrin have been determined to be expressed in many cancer cells and are each associated with negative prognostics of the disease. Accordingly, in some embodiments of the method, the cell is a cancer cell. In some embodiments, the cancer is a pancreatic cancer cell, a colon cancer cell, or a breast cancer cell.

In some embodiments, the cell is contacted with the NR4A1 antagonist ligand in vitro, such as in a cell or tissue culture context. In other embodiments, the cell is contacted in vivo. In such embodiments, the NR4A1 antagonist and/or inhibitor is administered to a subject according to any acceptable protocol. It will be appreciated that the antagonist and/or inhibitor is administered in an effective amount, as appropriate for the circumstances (such as for therapeutic effectiveness). Additionally, the antagonist can be administered with an acceptable carrier, as are well-known in the art.

Those having ordinary skill in the art will be able to ascertain the most effective dose and times for administering the agents (NR4A1 antagonists), considering route of delivery, metabolism of the compound, and other pharmacokinetic parameters such as volume of distribution, clearance, age of the subject, and so on. For example, the NR4A1 antagonist can be administered in any well-known method, such as by topical administration, oral administration, intravenous injection, intraperitoneal injection, intramuscular injection, intranasal administration, transdermal administration, rectal administration, or by any means which delivers an effective amount of the active agent to the tissue or site to be treated. Suitable dosages are those which achieve the desired endpoint. It will be appreciated that different dosages may be required for treating different disorders. An effective amount of an agent is, for example, that amount which causes a cessation or significant decrease in neoplastic cell count, growth, size, cell migration or cell invasion.

The agents (NR4A1 antagonists) can be administered along with a pharmaceutical carrier and/or diluent. The agents may also be administered in combination with other agents, for example, in association with other chemotherapeutic or immunostimulating drugs or therapeutic agents. Examples of pharmaceutical carriers or diluents useful in the present invention include any physiological buffered medium, i.e., about pH 7.0 to 7.4 comprising a suitable water soluble organic carrier. Suitable water soluble organic carriers include, but are not limited to corn oil, dimethylsulfoxide, gelatin capsules, and so on.

The subject can be any animal, such as a mammal, bird, reptile, or fish. Exemplary mammalian categories include rodents, primates, canines, felines, ungulates, lagomorphs, and the like. For example, the subject can be a human, monkey, ape or other primate, mouse, rat or other rodent, dog, cat, pig, horse, cow, or rabbit, etc.

In another aspect, the inhibition of β1-integrin as described herein is part of a method of treating a disorder or condition in a subject that is treatable by inhibiting β1-integrin.

As used herein, the term “treatment” means providing an ameliorative, curative, or preventative effect on the disorder or condition. In some embodiments, treatment includes preventing the escalation or progression, or slowing the rate of escalation or progression, of the condition (as compared to no or other treatment). In the context of cancers (more described below), treatment includes slowing or preventing the cell growth or rate of cell division, slowing or preventing cell migration, and/or slowing or preventing cell invasion.

The disorder can be any condition that is known where β1-integrin function and/or overexpression is known to play a role. As described herein, β1-integrin is critical for myriad cell functions, and overexpression of β1-integrin is associated with various cellular dysfunctions and diseases. Accordingly, various direct β1-integrin inhibitors have been addressed in numerous late stage clinical trials (see, e.g. Goodman and Picard, “Integrins as therapeutic targets,” Trends in Pharmacological Sciences, 33(7): (2012), incorporated herein by reference). Exemplary conditions include cancer, thrombosis, colitis, Crohn's disease, inflammatory bowel disease, multiple sclerosis, rheumatoid arthritis immunosuppression disorder, arthritis, asthma, stroke, restenosis, rhinitis, and osteoporosis. Exemplary cancers include pancreatic, colon, and breast cancer.

It will be understood that any embodiment, characteristic, element, definition, or general description provided for any aspect of the disclosure can be applied to any other aspect of the disclosure without limitation, unless explicitly stated. Thus, any embodiment discussed herein can be implemented with respect to any method, agent, or composition of the invention, and vice versa. Furthermore, agents and compositions of the invention can be used to achieve methods of the invention.

The use of the word “a” or “an,” when used in conjunction with the term “comprising” herein can mean “one,” but it is also consistent with the meaning of “one or more,” “at least one,” and “one or more than one.”

The use of the term “or” is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. As an alternative to or in addition to “comprising,” any embodiment herein can recite “consisting of.” The transitional phrase “consisting of” excludes any element, step, or ingredient not specified in the claim. Words using the singular or plural number also include the plural and singular number, respectively. Additionally, the words “herein,” “above,” and “below,” and words of similar import, when used in this application, shall refer to this application as a whole and not to any particular portions of the application.

Publications cited herein and the subject matter for which they are cited are hereby specifically incorporated by reference in their entireties.

The following is a description of the discovery of NR4A1 as a regulator of β1-integrin expression.

Rationale/Introduction

Pancreatic cancer is a devastating disease that is characterized by late diagnosis, extremely poor survival, limited chemotherapeutic options, and the development of drug resistance. Integrin signaling pathways in cancer are highly complex and are dependent on relative expression of individual integrin receptors and ligands, immediate downstream targets (e.g. FAK), and interactions with other pathways. β1-integrin is overexpressed in pancreatic tumors and β1-integrin silencing by RNA interference (RNAi) in pancreatic cancer cells decreases cell adhesion, migration and invasion, and downregulation of genes such as MMP-2 and MMP-9. Moreover, knockdown of β1-integrin in Colo-357 cells by RNAi also decreases expression of α2-, α3-, α5- and αv-integrins; these same integrins form heterodimers with β1-integrin and bind collagen (α2), laminin (α3), and fibronectin (α5 and αv). β1-integrin silencing by RNA interference (RNAi) also decreases tumor growth and metastasis, and similar results have been observed in vivo using β1-integrin antibodies. Rip1Tag2 transgenic mice express the Simian Virus 40 large T-antigen under the control of the rat insulin promoter and spontaneously develop pancreatic β-cell tumors, and ablation of β1-integrin in β-tumor cells decreases tumor cell growth and metastasis. Pancreatic cancer cells express β1- and other α-integrin partners and, not surprisingly, β1-integrin heterodimers are activated by multiple extracellular matrix proteins (e.g. collagens, fibronectin, laminin, vitronectin) and many other extracellular factors, including tissue factor (TF) and alternatively spliced TF (asTF) which are overexpressed in pancreatic tumors and exhibit potent growth promoting, angiogenic, and metastatic activity. The L1 cell adhesion molecule (L1CAM) is a transmembrane glycoprotein (200-220 kDA) that is overexpressed in pancreatic ductal adenocarcinoma cells. L1CAM-mediated drug-resistance, survival, and epithelial to mesenchymal transition (EMT) in pancreatic cancer cells is due to interactions with α5 and β1-integrins and the immediate downstream targets include FAK, integrin-linked kinase (ILK) and PI3-K. L1CAM-β1-integrin activation is associated with induction of IL-1β and activation of NFκB, and this signaling pathway supports a motile and invasive tumor cell phenotype. Other factors including neuropilin-1, glial cell line-derived neurotrophic factor, thrombin, Snail, Slug and interleukin β either interact with or enhance β1-integrin and thereby modulate downstream signaling in pancreatic cancer cells.

Accordingly, β1-integrin is overexpressed in pancreatic tumors and is a negative prognostic factor, and this correlates with the pro-oncogenic functions of β1-integrin receptor-mediated migration, invasion and drug resistance, demonstrating the importance of β1-integrin as a drug target for treating pancreatic cancer.

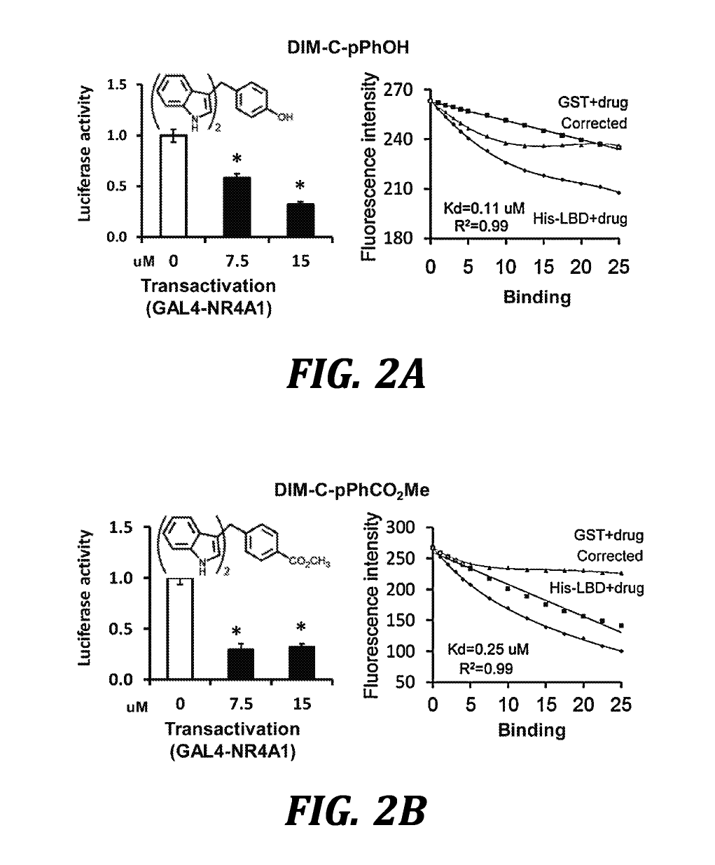

The orphan nuclear receptor NR4A1 (TR3) and related receptors [(NR4A2 (Nurr1) and NR4A3 (Nor1)] are immediate early genes induced by multiple stimuli/stressors and play essential roles in metabolic processes, inflammation, vascular function, steroidogenesis, and the central nervous system. NR4A1 is also overexpressed in multiple tumors and cancer cell lines. In pancreatic cancer patients, NR4A1 levels are higher in tumor vs. non-tumor tissues. Knockdown or overexpression of NR4A1 in pancreatic and solid tumor-derived cell lines indicates that NR4A1 is pro-oncogenic and regulates cancer cell proliferation, survival, migration/invasion and metastasis. Several pro-apoptotic anticancer drugs that do not bind NR4A1 induce nuclear translocation of NR4A1 which subsequently binds mitochondrial bcl-2 to form a pro-apoptotic complex. Our studies show that a series of 1,1-bis(3′-indolyl)-1-(p-substituted phenyl) methane (C-DIM) analogs (FIG. 1) inactivate nuclear NR4A1 to inhibit growth and induce apoptosis in pancreatic and other cancer cell lines. Results of RNAi in pancreatic cancer cells show that NR4A1 regulates expression of pro-survival genes such as survivin and bcl-2 through interactions with Sp1 bound to GC-rich cis-elements, and NR4A1-dependent regulation of thioredoxin domain-containing protein 5 (TXNDC5) maintains low stress levels in pancreatic and other cancer cells. NR4A1 also binds and inactivates p53 and, in lung cancer cells, this results in activation of mTOR (FIG. 1). This pro-oncogenic pathway is less relevant for pancreatic cancers which primarily express mutant p53. Thus, transfection of pancreatic cancer cells with siNR4A1 or treatment with the phydroxyphenyl C-DIM analog (DIM-C-pPhOH) (FIGS. 1, 2A, and 2B) that inactivates NR4A1 downregulates survival genes and induces oxidative and endoplasmic reticulum stress, resulting in apoptosis.

However, the NR4A1-regulated genes and pathways associated with migration and invasion of pancreatic cancer cells have not been identified. Therefore, the effects of NR4A1 silencing were investigated using a Sentrix Human V.3 HT12 beadchip array and analysis of genes involved in migration and invasion showed that β1-integrin is an NR4A1-regulated gene. These results, coupled with results of assays employing NR4A1 antagonists, demonstrate that NR4A1 antagonists decrease expression of β1-integrin in pancreatic cancer cells and establish the NR4A1 antagonists as a novel class of mechanism-based anticancer agents for treating this disease, or any other disease characterized by β1-integrin expression or overexpression.

Results

(a) C-DIMs as NR4A1 Ligands and Antagonists.

C-DIMs, such as DIM-C-pPhOH, have been shown to inactivate NR4A1. These compounds bind the receptor and are receptor antagonists. Thus, C-DIMs are the first NR4A1 antagonists that inhibit NR4A1-mediated pro-oncogenic genes/pathways. The transactivation and NR4A1 binding of the p-hydroxyphenyl (DIM-C-pPhOH) analog was initially investigated. DIM-C-pPhOH was shown to inactivate NR4A1 and give results similar to knockdown of NR4A1 (siNR4A1) in pancreatic and lung cancer cell lines was investigated. Panc1 cells were transfected with a GAL4-NR4A1 (full length) construct and a UAS₅-luc reporter system which contains 5 GAL4 response elements, and treatment with DIM-CpPhOH decreased luciferase activity (FIG. 2A, left panel). Similar results were observed in Panc1 cells transfected with an NBRE-luc reporter system that contains three tandem NBREs that are bound by the NR4A1 monomer (not shown). A fluorescence quenching assay and incubated selected C-DIMs with a His-tagged protein containing the ligand binding domain (LBD) of NR4A1 (i.e. His-NR4A1 (LBD)]. The results show that DIM-C-pPhOH quenches fluorescence (FIG. 2A, right panel), indicating for the first time that C-DIMs physically bind NR4A1. Moreover, previous studies show that DIM-C-pPhOH-mediated inactivation of NR4A1 is nuclear, indicating that this compound is an NR4A1 antagonist. Additional assays show that the p-carboxymethylphenyl (DIM-C-pPhCO₂Me) also inactivates NR4A1 and this compound inhibits NR4A-dependent transactivation and binds the LBD of NR4A1 in a fluorescence binding assay (FIG. 2B).

(b) Pro-Oncogenic Function of NR4A1 in Pancreatic Cancer.

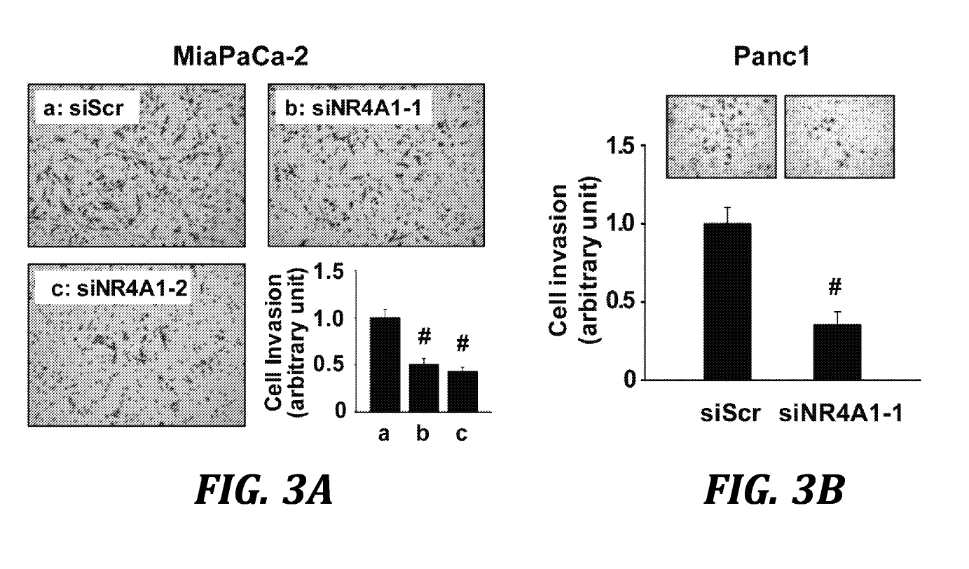

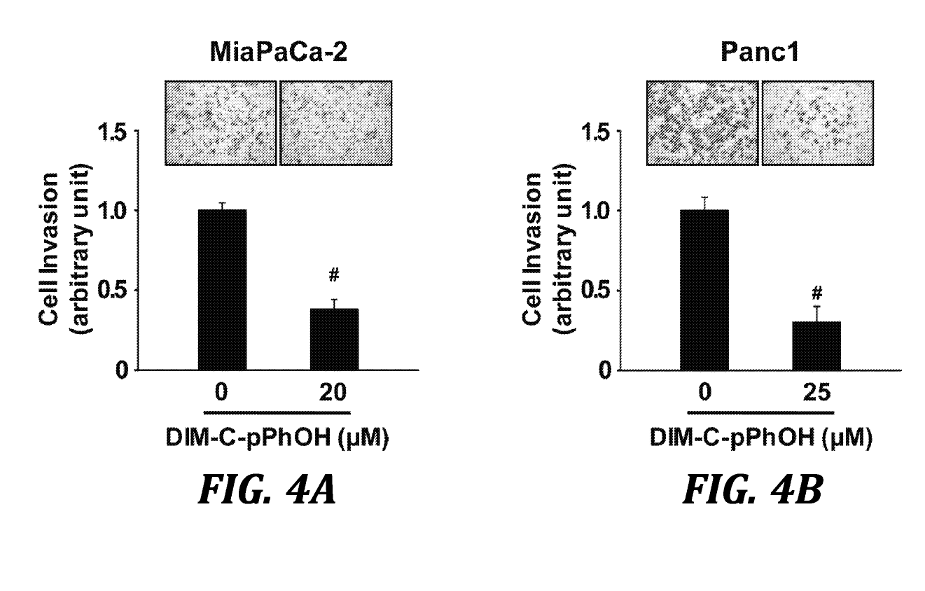

Previous studies indicate that in pancreatic cancers, NR4A1 regulates pro-survival genes (e.g. survivin) through an Sp1/p300/NR4A1 complex in which Sp1 binds promoter DNA. It was recently demonstrated that NR4A1 maintains levels of oxidative and endoplasmic reticulum (ER) stress by regulating expression of thioredoxins (see FIG. 1). Both of these pathways regulate survival and cell proliferation, and siNR4A1 or DIM-CpPhOH inhibit pancreatic cancer cell growth and induce apoptosis. A third pathway involves activation of mTOR which is due to inactivation of p53 by binding NR4A1 and this pathway is only observed in cancer cell lines and tumors expressing wild-type p53. Knockdown of NR4A1 in MiaPaCa-2 cells by two different oligonucleotides (siNR4A1-1 and siNR4A1-2) not only decreased cell growth and induced apoptosis (data not shown) but also decreased cancer cell invasion in a Boyden Chamber assay (FIG. 3A). Similar results were observed in Panc1 cells transfected with one of the siNR4A1 oligonucleotides. A similar approach was used for the NR4A1 antagonist DIM-C-pPhOH (20 μM) which also decreased invasion in pancreatic cancer cells (FIGS. 4A and 4B), demonstrating that the pro-oncogenic functions of NR4A1 facilitate cancer cell invasion which can be inhibited by NR4A1 antagonists.

(c) Pro-Oncogenic Pathways/Genes Regulated by NR4A1.

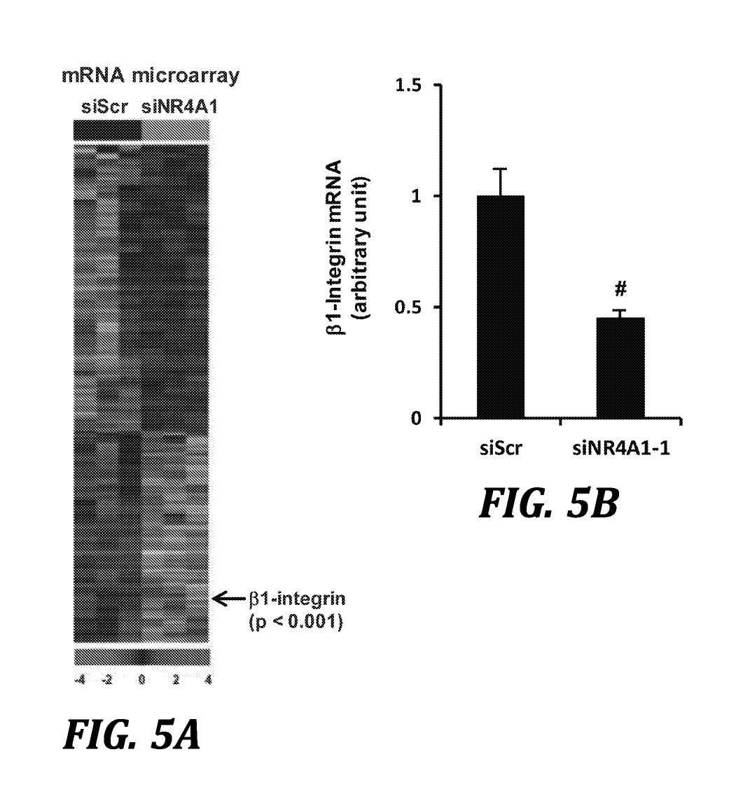

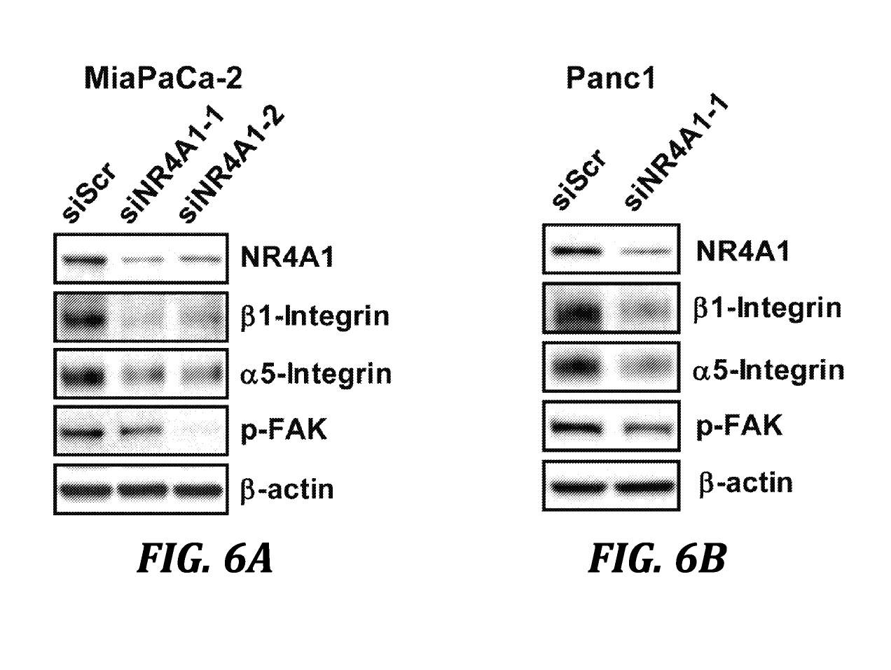

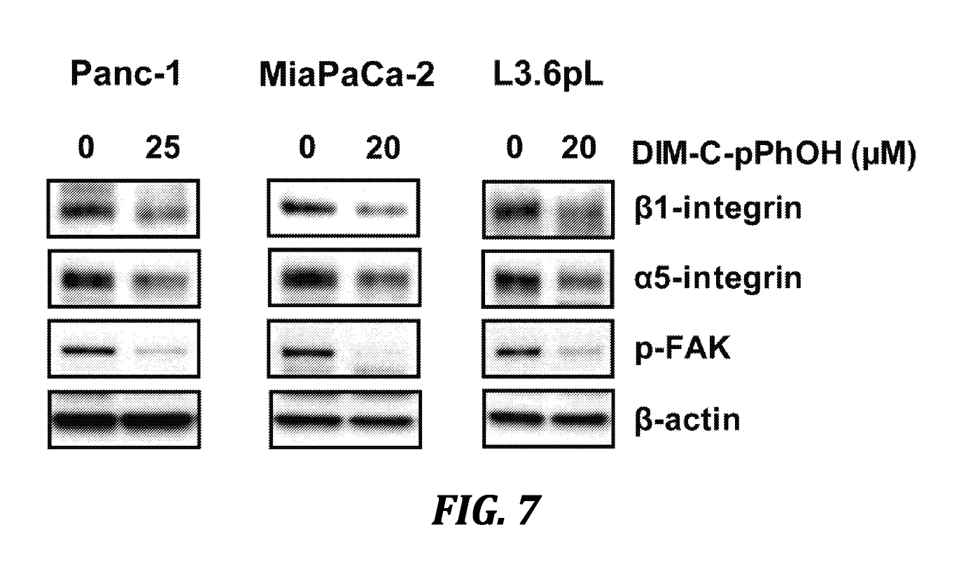

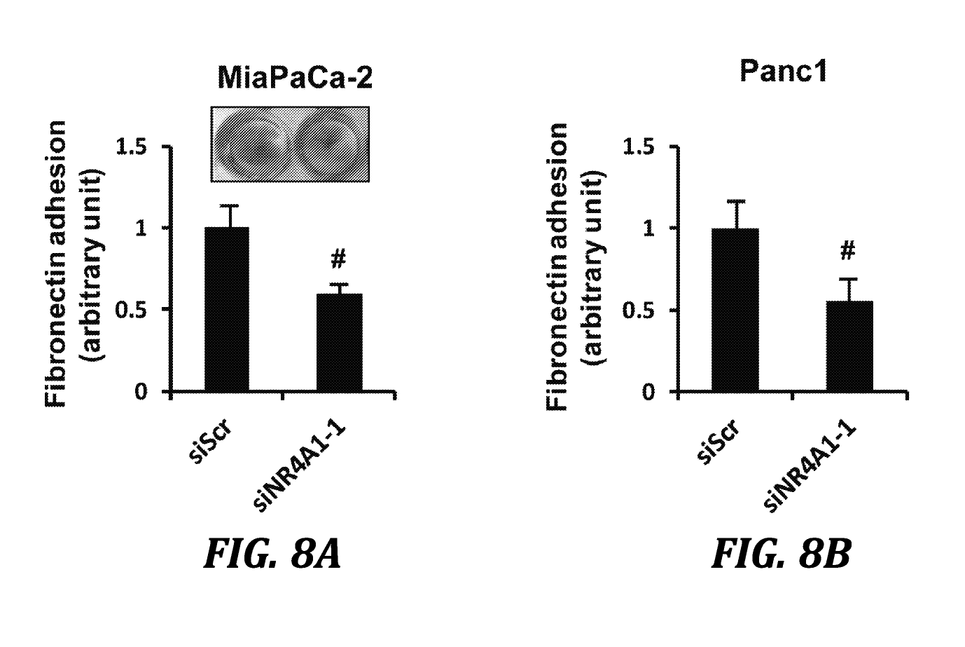

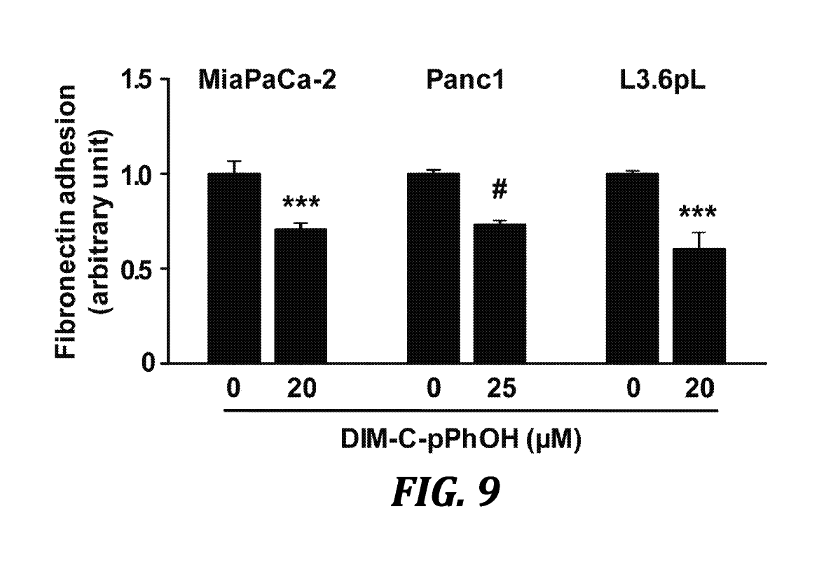

Discovery of NR4A1-regulated pro-invasion/migration genes was determined using Illumina Human HT-12V4 Beadchip arrays (FIGS. 5A and 5B). Knockdown of NR4A1 in Panc1 cells decreased expression of β1-integrin which is known to play an important role in cell migration and invasion. Knockdown of NR4A1 with siNR4A1-1 and siNR4A1-2 in MiaPaCa-2 cells decreased expression of the receptor and this was accompanied by decreased levels of β1-integrin, α5-integrin and phosphorylation of the downstream kinase (p-FAK) (FIG. 6A). Similar results were observed in Panc1 cells (FIG. 6B), demonstrating that NR4A1 regulates β1-integrin expression. The loss of α5-integrin may be indirect since it was previously reported that β1-integrin silencing by RNAi decreased α5-integrin. FIG. 7 confirms that DIM-C-pPhOH also decreased expression of β1-integrin and related gene products. β1-integrin interacts with extracellular matrix (ECM) proteins, and results in FIGS. 8A and 8B show that MiaPaCa-2 and Panc1 cell adhesion to fibronectin (an ECM protein) was significantly decreased after knockdown of NR4A1. Decreased adhesion to fibronectin-coated plates was also observed in pancreatic cancer cells treated with DIM-C-pPhOH (FIG. 9), confirming that siNR4A1 and NR4A1 antagonists decrease expression of β1-integrin. This represents a novel approach for targeting this pro-oncogenic factor in pancreatic cancer.

(d) In Vivo Studies.

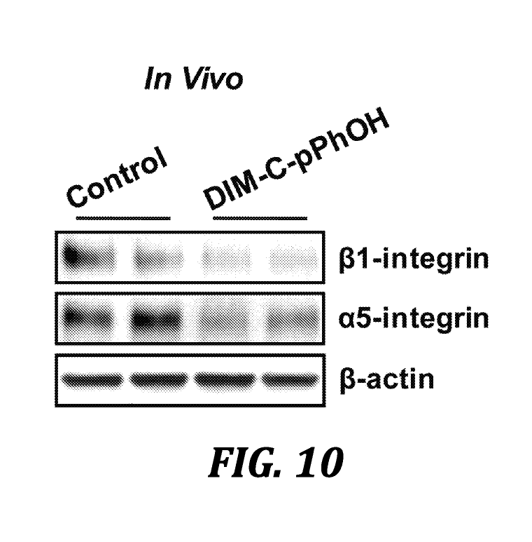

Previous studies show that DIM-CpPhOH inhibits tumor growth in athymic nude mice in an orthotopic model. In the present study, tumor lysates from these athymic nude mice were analyzed by Western blots demonstrating that DIM-C-pPhOH decreased expression of β1- and α5-integrin. These results complement the in vitro, described above, studies showing that the NR4A1 antagonist decreases β1-integrin, and this response, coupled with the effects of NR4A1 knockdown or NR4A1 antagonists on other NR4A1-regulated pathways (FIG. 10), indicate the clinical potential of NR4A1 antagonists for pancreatic cancer chemotherapy.

(e) Receptor Binding of Various C-DIMs.

Using a fluorescent binding assay, a library of C-DIM compounds were screened. Specifically, 14 different C-DIM analogs with pPhX on the bridging carbon (i.e., as R₈) were screened, where X was CF₃, Br, F, t-Butyl, OCH₃, N-dimethylamino, H, OH, C₆H₅, CN, CH₃, Cl, I and CO₂Me. Each analog bound the ligand binding domain of NR4A1 and K_{D} values for binding varied from 0.1-0.71 μM.

(f) Analysis of NR4A1 Regulation of β1-Integrin in Other Cancer Cell Lines.

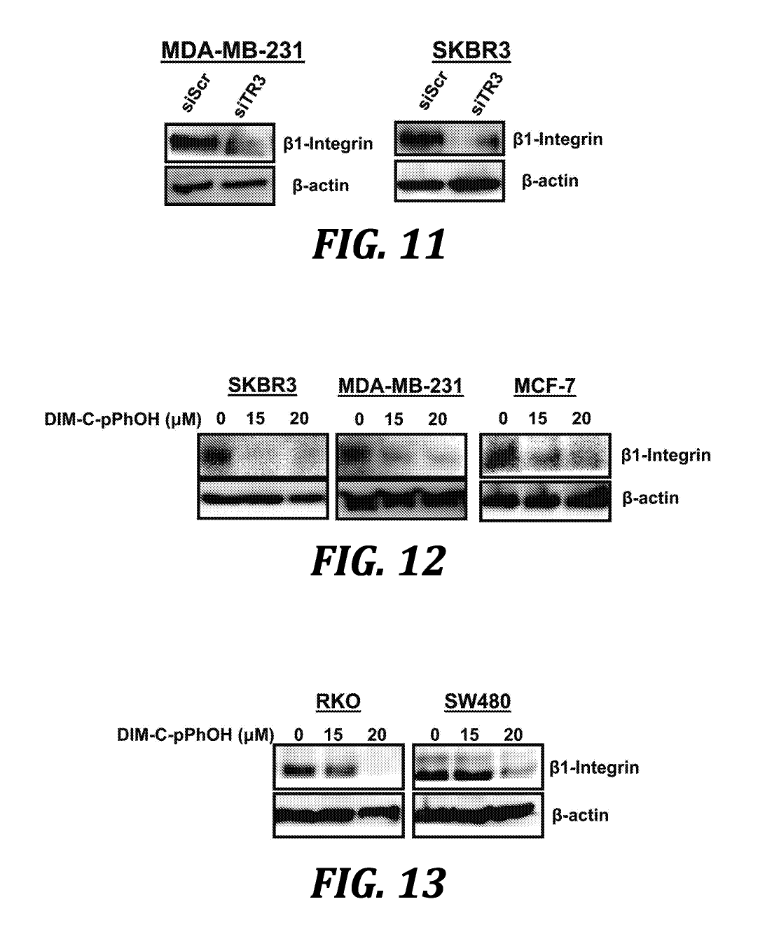

FIG. 11 demonstrates that knockdown of NR4A1 (TR3) in human breast cancer MDA-MB-231 cells and SKBR3 cells by RNA interference results in decreased expression of β1-integrin. Similar results were also observed in MCF-7 breast cancer cells (not shown). Treatment of these same three breast cancer cell lines with the NR4A1 antagonist DIM-C-pPhOH also decreased β1-integrin expression in each cell line (see FIG. 12). Similar results were also observed in SW480 and RKO colon cancer cells treated with the same NR4A1 C-DIM antagonist (see FIG. 13).

(g) Analysis of Downstream Effects of NR4A1/β1-Integrin Inhibition.

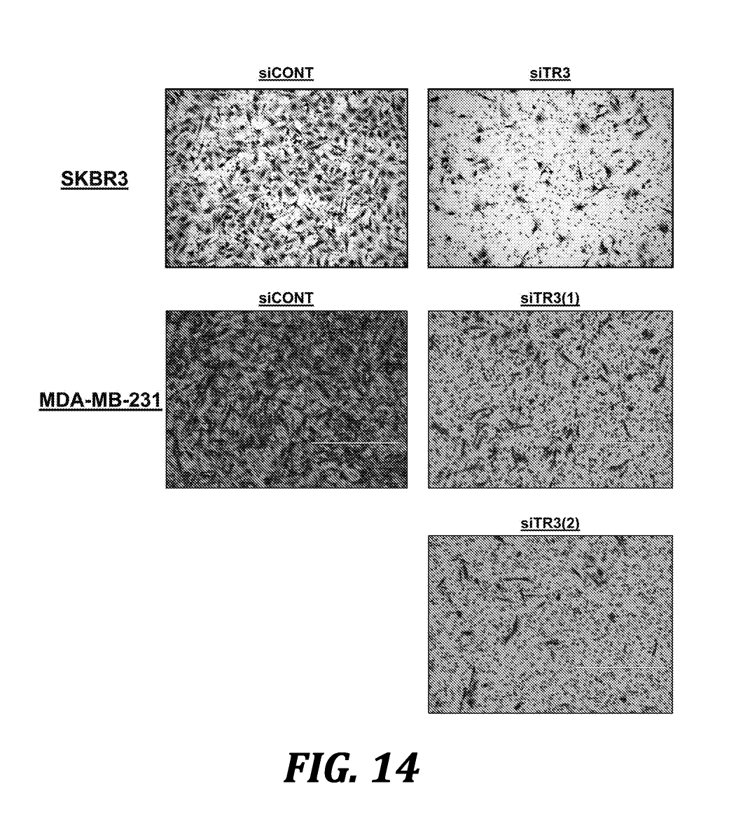

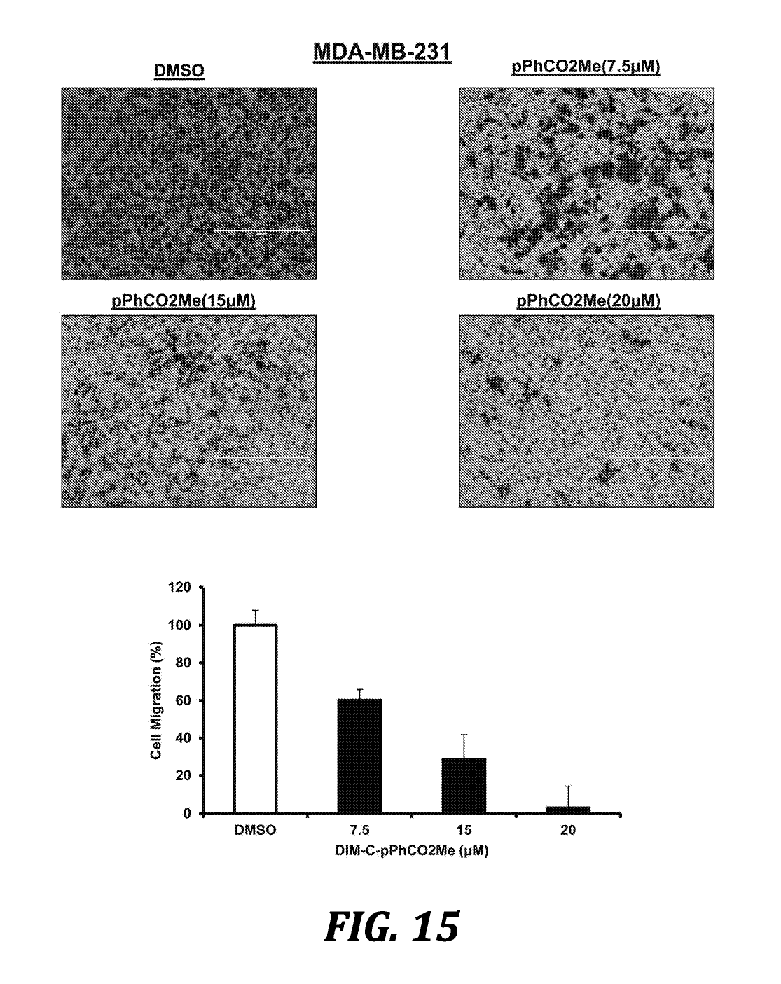

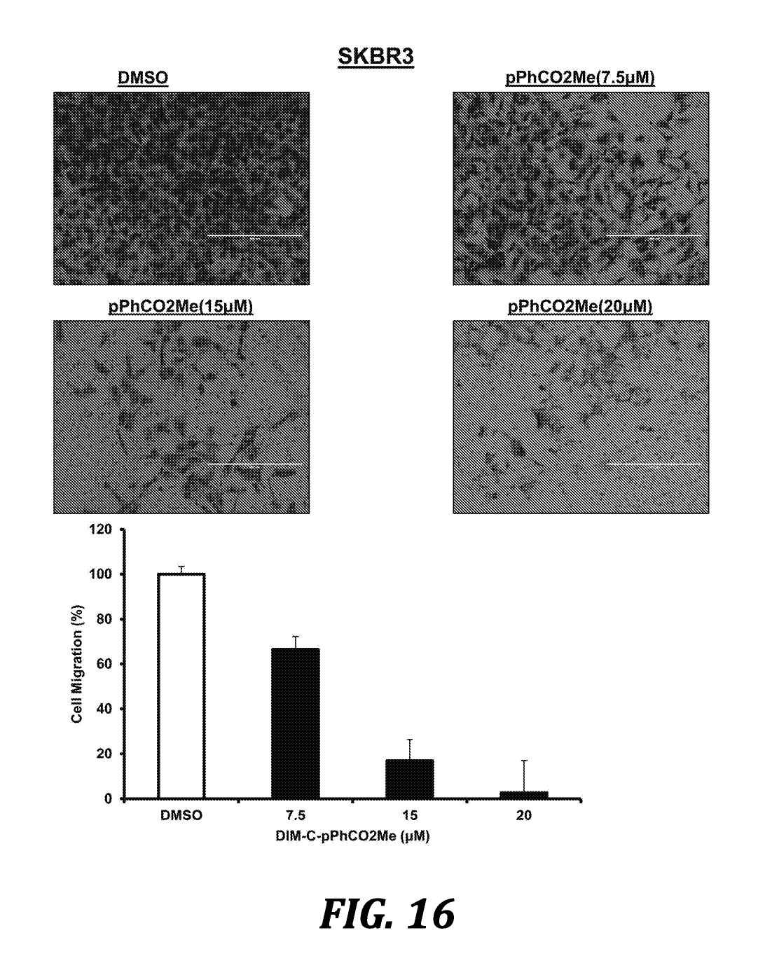

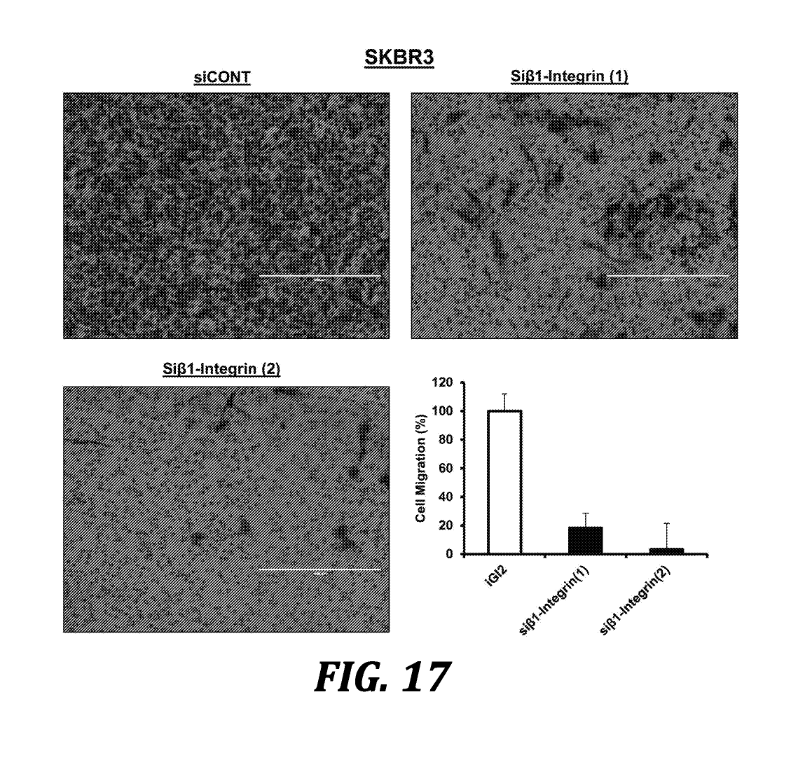

Assessing downstream effects of the above β1-integrin inhibition, knockdown of NR4A1 (TR3) by RNA interference decrease migration and invasion performance of SKBr3 and MDA-MB-231 cells (FIG. 14). Similar results were observed from the administration of NR4A1 antagonist DIM-CpPhOH (data not shown). Moreover, the effects of other NR4A1 antagonists on migration and invasion of the breast cancer cells were investigated. FIGS. 15 and 16 demonstrate that DIM-C-pPhCO₂Me inhibits migration and invasion of MDA-MB-231 cells and SKBR3 breast cancer cells, respectively. Similar results were observed after knockdown of NR4A1 by RNA interference (see FIG. 14). FIG. 17 shows that in SKBR3 cells knockdown of β1-integrin by RNA interference also decreases cell migration and invasion. These results indicate similar response pathways in three different cancer cell types (i.e. pancreatic, breast and colon) where NR4A1 regulates β1-integrin, and expression of this pro-oncogenic factor can be decreased with NR4A1 antagonists.

Accordingly, it is demonstrated that NR4A1 regulates the expression of β1-integrin in various cell lines, and that inhibition of NR4A1 results in the inhibition of β1-integrin expression and its downstream effects, such as its pro-oncogenic and pro-metastatic effects manifesting in cancer cell migration and invasion. Accordingly, these data demonstrate that NR4A1 inhibitors/antagonists represent a novel and useful tool to target β1-integrin-associated disorders, including pancreatic, colon, and breast cancer.

While the preferred embodiment of the invention has been illustrated and described, it will be appreciated that various changes can be made therein without departing from the spirit and scope of the invention.

1. A method of inhibiting β1-integrin expression in a cell, comprising inhibiting NR4A1 in the cell.

2. The method of claim 1, wherein inhibiting NR4A1 in the cell comprises reducing the level of functional NR4A1 in the cell.

3. The method of claim 1, wherein inhibiting NR4A1 in the cell comprises contacting the cell with an NR4A1 ligand that exhibits antagonist activity.

4. The method of claim 3, wherein the NR4A1 antagonist is a C-substituted diindolylmethane compound (C-DIM).

5. The method of claim 4, wherein the C-DIM is 1,1-bis(3′-indolyl)-1-(p-hydroxyphenyl)methane (DIM-C-pPhOH) or a related compound where the -pPhOH group contains other substituents or is replaced by another aromatic group.

6. The method of claim 1, wherein the cell is a cancer cell.

7. The method of claim 6, wherein the cell is a pancreatic cancer cell, a colon cancer cell, or a breast cancer cell.

8. The method of claim 3, wherein the cell is contacted with the NR4A1 antagonist in vitro.

9. The method of claim 3, wherein the cell is contacted with the NR4A1 antagonist in vivo by administering an effective amount of the NR4A1 antagonist to a subject.

10. The method of claim 9, wherein the NR4A1 antagonist is administered with a pharmaceutically acceptable carrier.

11. The method of claim 9, wherein the administering comprises topical administration, oral administration, intravenous injection, intraperitoneal injection, intramuscular injection, intranasal administration, transdermal administration, or rectal administration.

12. The method of claim 9, wherein the subject is a mammal.

13. A method of treating a disorder treatable by inhibiting β1-integrin in one or more cells of a subject with the disorder, comprising administering a therapeutically effective amount of an NR4A1 inhibitor to the subject.

14. The method of claim 13, wherein the NR4A1 inhibitor comprises a ribonucleic acid that corresponds to at least a portion of the NR4A1 mRNA sequence set forth in SEQ ID NO:1.

15. The method of claim 13, wherein the NR4A1 inhibitor is an NR4A1 antagonist.

16. The method of claim 15, wherein the NR4A1 antagonist is a C-substituted diindolylmethane compound (C-DIM).

17. The method of claim 16, wherein the C-DIM is 1,1-bis(3′-indolyl)-1-(p-hydroxyphenyl)methane (DIM-C-pPhOH) or a related compound where the -pPhOH group contains other substituents or is replaced by another aromatic group.

18. The method of claim 17, wherein the disorder is cancer, thrombosis, colitis, Crohn's disease, inflammatory bowel disease, multiple sclerosis, rheumatoid arthritis immunosuppression disorder, arthritis, asthma, stroke, restenosis, rhinitis, and osteoporosis.

19. The method of claim 18, wherein the cancer is a pancreatic cancer, a colon cancer, or a breast cancer.

20. The method of claim 19, wherein treating the cancer comprises preventing or inhibiting the migration capacity of cancer cells in the subject.

21. The method of claim 19, wherein treating the cancer comprises preventing or inhibiting the invasion capacity of cancer cells in the subject.

22. The method of claim 19, wherein treating the cancer comprises preventing or inhibiting the growth of cancer cells in the subject.

23. The method of claim 13, wherein the subject is a mammal selected from the group consisting of rat, mouse, guinea pig, dog, cat, cow, horse, sheep, pig, primate, and human.

24. The method of claim 13, wherein the NR4A1 inhibitor is administered with a pharmaceutically acceptable carrier.

25. The method of claim 13, wherein the administering comprises topical administration, oral administration, intravenous injection, intraperitoneal injection, intramuscular injection, intranasal administration, trans dermal administration, or rectal administration.


SEQUENCE LISTING (1 sequence)

SEQ ID NO: 1 - DNA, 2603 bp, Homo sapiens
ttcctggtgtaagctttggtatggatggtggccgtctccctacagactgggagctgttagagggcagggatcctagctgacacatctatgtcctcgccttggttggaggcctccaccatggacagaggccaggccctgcccctcccaggcagcctggctccttctgctgggccctgaaggcagacgggataatgtggttggccaaggcctgttggtccatccagagtgagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaactgaaaaaaaaaaaaaa